(19) United States
(12) Patent Application Publication
(10) Pub. No.: US 2024/0197199 A1
(43) Pub. Date: Jun. 20, 2024

(54) TACTILE AND AUDITORY RESPIRATION BIOFEEDBACK WITH A WEARABLE DEVICE

(71) Applicant: Oura Health Oy, Oulu (FI)

(72) Inventors: Olli Petteri Heikkinen, Oulu (FI); Heli Tuulia Koskimäki, Oulu (FI)

(21) Appl. No.: 18/068,353

(22) Filed: Dec. 19, 2022

(51) Int. Cl. A61B 5/08; A61B 5/00; A61B 5/0295

(52) U.S. Cl. CPC A61B 5/0816; A61B 5/0295; A61B 5/486; A61B 5/742

(57) ABSTRACT

Methods, systems, and devices that support techniques for tactile and auditory respiration biofeedback are described. A method may include acquiring physiological data associated with a user via a wearable device during a respiration biofeedback session, the physiological data including at least respiration rate data. The method may include generating, via a user device during the respiration biofeedback session, a set of tactile vibration pulses, a set of audio pulses, or both, where the set of tactile vibration pulses, the set of audio pulses, or both, are generated in accordance with a respiration rate pattern that is based on the respiration rate data. The method may include causing a graphical user interface (GUI) of the user device to display an indication of one or more changes in the physiological data acquired during the respiration biofeedback session.

### FIELD OF TECHNOLOGY

The following relates to wearable devices and data processing, including techniques for tactile and auditory respiration biofeedback using wearable devices.

### BACKGROUND

Some wearable devices may be configured to collect data from users associated with motion data, temperature data, heart rate data, photoplethysmography (PPG) data, bioimpedance data, respiration rate data, and the like. Some systems associated with the wearable devices may also be able to perform various actions, such as providing certain health insights to users.

### BRIEF DESCRIPTION OF THE DRAWINGS

FIG. 1 illustrates an example of a system that supports techniques for tactile and auditory respiration biofeedback in accordance with aspects of the present disclosure.

FIG. 2 illustrates an example of a system that supports techniques for tactile and auditory respiration biofeedback in accordance with aspects of the present disclosure.

FIG. 3 illustrates an example of a system that supports techniques for tactile and auditory respiration biofeedback in accordance with aspects of the present disclosure.

FIG. 4 illustrates an example of a respiration rate pattern that supports techniques for tactile and auditory respiration biofeedback in accordance with aspects of the present disclosure.

FIG. 5 illustrates an example of a graphical user interface (GUI) that supports techniques for tactile and auditory respiration biofeedback in accordance with aspects of the present disclosure.

FIG. 6 illustrates a block diagram of an apparatus that supports techniques for tactile and auditory respiration biofeedback in accordance with aspects of the present disclosure.

FIG. 7 illustrates a block diagram of a wearable device manager that supports techniques for tactile and auditory respiration biofeedback in accordance with aspects of the present disclosure.

FIG. 8 illustrates a diagram of a system including a device that supports techniques for tactile and auditory respiration biofeedback in accordance with aspects of the present disclosure.

FIG. 9 illustrates a flowchart showing methods that support techniques for tactile and auditory respiration biofeedback in accordance with aspects of the present disclosure.

### DETAILED DESCRIPTION

Wearable devices may be used to collect, monitor, and track physiological data associated with a user based on sensor measurements performed by the wearable device. Examples of physiological data may include temperature data, heart rate data, photoplethysmography (PPG) data, bioimpedance data, and the like. The physiological data collected, monitored, and tracked via the wearable device may be used to gain health insights about the user, such as the user's sleeping patterns, activity patterns, and the like. In some cases, physiological data collected by a wearable device may include respiration rate data that is used to determine a respiration rate (e.g., a breathing rate) of the user.

Conventional wearable devices may be configured to collect respiration data to determine when a user is breathing at an elevated respiration rate. For example, an elevated respiration rate may be used to determine when the user is participating in various activities, such as walking, running, swimming, or the like thereof. Additionally, or alternatively, an elevated respiration rate may be used to determine a physiological state of the user, such as if the user is stressed or anxious. However, while wearable devices may be able to determine and display a user's respiration rate, some wearable devices may be unable to assist the user in reducing their respiration rate. For instance, the wearable device may alert the user of an elevated respiration rate but the user may be unable to determine how to reduce the respiration rate.

Accordingly, aspects of the present disclosure are directed to techniques that leverage physiological data, such as respiration data, collected via wearable devices, and vibrational capabilities, auditory capabilities, or both, of a user device to support user relaxation. In particular, aspects of the present disclosure may utilize respiration rate data collected during a respiration biofeedback session (e.g., relaxation session, meditation session) to determine a respiration rate pattern in order to generate a set of tactile vibration pulses (e.g., haptic feedback), a set of audio pulses, or both, to guide a user to adjust (e.g., reduce) a respiration rate of the user. By reducing the respiration rate of the user, aspects of the present disclosure may aid in relaxation of the user, and may therefore result in improved scores associated with the user (e.g., Sleep Score, Readiness Score), improved physiological conditions of the user, improved psychological state of the user, or the like thereof.

For example, a system may support a respiration biofeedback mode that a user may initiate a respiration biofeedback session via a user device. During the respiration biofeedback session, the system may acquire physiological data associated with the user, the physiological data including at least respiration rate data. The system may determine a first respiration rate associated with the user based on the respiration rate data, where the first respiration rate includes an inhalation rate of the user, an exhalation rate of the user, or both.

In some examples, the system may determine a respiration rate pattern based on the first respiration rate. That is, the respiration rate pattern may be an artificial respiration rate pattern, including an inhalation pattern and an exhalation pattern, that is lower (e.g., slower) than the first respiration rate of the user. Additionally, or alternatively, the system may determine a respiration rate pattern based on a respiration rate of another user, a predefined respiration rate pattern, or the like thereof. For example, the system may receive respiration rate data associated with another user (e.g., a yoga instructor) and may determine a respiration rate pattern based on the respiration rate data associated with the other user. In another example, the system may receive, from a meditation application, an indication of a predefined respiration rate pattern associated with a meditation session.

Subsequently, the system may generate (e.g., via the user device, the wearable ring device, or both), during the respiration biofeedback session, a set of tactile vibrational pulses (e.g., vibration pulses), a set of audio pulses (e.g., sound pulses), or both, in accordance with the respiration rate pattern (e.g., inhalation pattern and exhalation pattern). That is, the system may instruct the user to inhale and exhale according to the respiration rate pattern via the set of tactile vibration pulses, the set of audio pulses, or both. For example, the system may instruct the user to place their hand on the user device and may generate the set of tactile vibration pulses according to the respiration rate pattern, instructing the user to breathe according to the respiration rate pattern indicated via the set of tactile vibration pulses. In other words, the system may instruct the user to inhale during a first set of tactile vibration pulses generated according to the inhalation pattern and to exhale during a second set of tactile vibration pulses generated according to the exhalation pattern. In such cases, the first set of tactile vibration pulses may increase in frequency during the inhalation of the user and the second set of tactile vibration pulses may decrease in frequency during the exhalation of the user. The change in frequency may indicate to the user when to transition from inhalation to exhalation (e.g., and vice-versa).

Additionally, or alternatively, the system may generate the set of audio pulses according to the respiration rate pattern, instructing the user to breathe according to the respiration rate pattern generated via the set of audio pulses. In other words, the system may instruct the user to inhale during a first set of audio pulses generated according to the inhalation pattern and to exhale during a second set of audio pulses generated according to the exhalation pattern. The audio pulses may include beeps, chimes, or any other form of sound pulse that may be generated in accordance with the respiration rate pattern. In such cases, the first set of audio pulses may increase in frequency during inhalation of the user and the second set of audio vibrational pulses may decrease in frequency during exhalation of the user. The change in frequency may indicate to the user when to transition from inhalation to exhalation (e.g., and vice-versa).

In some examples, the system may update the respiration rate pattern associated with generation of the set of tactile vibration pulses, the set of audio pulses, or both. In other words, during the respiration biofeedback session, the respiration rate associated with the user may lower (e.g., become slower) such that the system may update the respiration rate pattern to aid in further lowering the respiration rate of the user. For example, the system may acquire additional physiological data, including additional respiration rate data, associated with the user via the wearable device during the respiration biofeedback session. The system may determine a second respiration rate associated with the user (e.g., the second respiration rate being lower than the first respiration rate), such that the system may determine a second respiration rate pattern, the second respiration rate pattern being associated with a second artificial respiration rate lower than the second respiration rate of the user. Accordingly, the system may generate, via the user device during the respiration biofeedback session, the set of tactile vibrational pulses, the set of audio pulses, or both, in accordance with the second respiration rate pattern.

Additionally, or alternatively, a respiration rate pattern based on a respiration rate of another user or a predefined respiration rate may be lower (e.g., much lower) than the respiration rate of the user, such that the system may incrementally update the respiration rate pattern associated with generation of the set of tactile vibration pulses, the set of audio pulses, or both, such that the respiration rate of the user may incrementally be lowered (e.g., may slowly be reduced). For example, the system may receive an indication of a predefined respiration rate pattern associated with a desired respiration rate, the first respiration rate being lower than the respiration rate of the user. As such, the system may determine a first respiration rate pattern associated with a first respiration rate, the first respiration rate being lower than the respiration rate of the user but higher than the desired respiration rate. Accordingly, the system may generate, via the user device during the respiration biofeedback session, a set of tactile vibrational pulses, a set of audio pulses, or both, in accordance with the first respiration rate pattern. Additionally, the system may monitor the respiration rate of the user such that the system may update the first respiration rate pattern to a second respiration rate pattern based on the respiration rate of the user being within a threshold of (e.g., close to or the same as) the first respiration rate (e.g., associated with the first respiration rate threshold). The second respiration rate pattern may be lower than the first respiration rate pattern but higher than the desired respiration rate pattern. Accordingly, the system may generate a set of tactile vibrational pulses, a set of audio pulses, or both, in accordance with the second respiration rate pattern. The system may monitor the respiration rate of the user and update the respiration rate pattern associated with generation of the set of tactile vibrational pulses, the set of audio pulses, or both, incrementally until the respiration rate pattern is within a threshold of the desired respiration rate pattern.

In some examples, the system may initiate the respiration biofeedback session based on a user input. That is, the user may manually initiate the respiration biofeedback session via the user device, the wearable device, or both. Additionally, or alternatively, the system may prompt the user to initiate the respiration biofeedback session. For example, the system may determine that a suggested (e.g., calculate) bedtime for the user is within a threshold duration of the current time and may prompt the user to initiate a respiration biofeedback session to aid in relaxing before bedtime. In another example, the system may acquire additional physiological data, such as a heart rate, a respiration rate, or one or more stress indicators, associated with the user and may prompt the user to initiate the respiration biofeedback session based on the additional physiological data. For example, the system may determine that the user is showing signs of stress (e.g., based on the additional physiological data), and may prompt the user to initiate a respiration biofeedback session to aid with stress reduction.

Additionally, or alternatively, the system may identify that another user connected with the user (e.g., spouse, training partner, fitness instructor) has initiated a respiration biofeedback session and may prompt the user to initiate a respiration biofeedback session (e.g., shared respiration biofeedback session) based on the other user. In other words, the system associated with the user may be connected to a system associated with the other user, such as via one or more applications, one or more paired wearable devices, one or more paired user devices, or the like thereof, and may prompt the user to initiate a respiration biofeedback session based on the other user initiating a respiration biofeedback session. In some examples, the respiration biofeedback session may be a shared (e.g., paired) respiration biofeedback session, such that a respiration biofeedback session associated with the user is the same (e.g., in sync) as a respiration biofeedback session associated with the other user. A shared respiration biofeedback session may include any quantity of users.

Aspects of the disclosure are initially described in the context of systems supporting physiological data collection from users via wearable devices. Aspects of the disclosure are then described in the context of vibrational patterns and an example GUI. Aspects of the disclosure are further illustrated by and described with reference to apparatus diagrams, system diagrams, and flowcharts that relate to techniques for tactile and auditory respiration biofeedback.

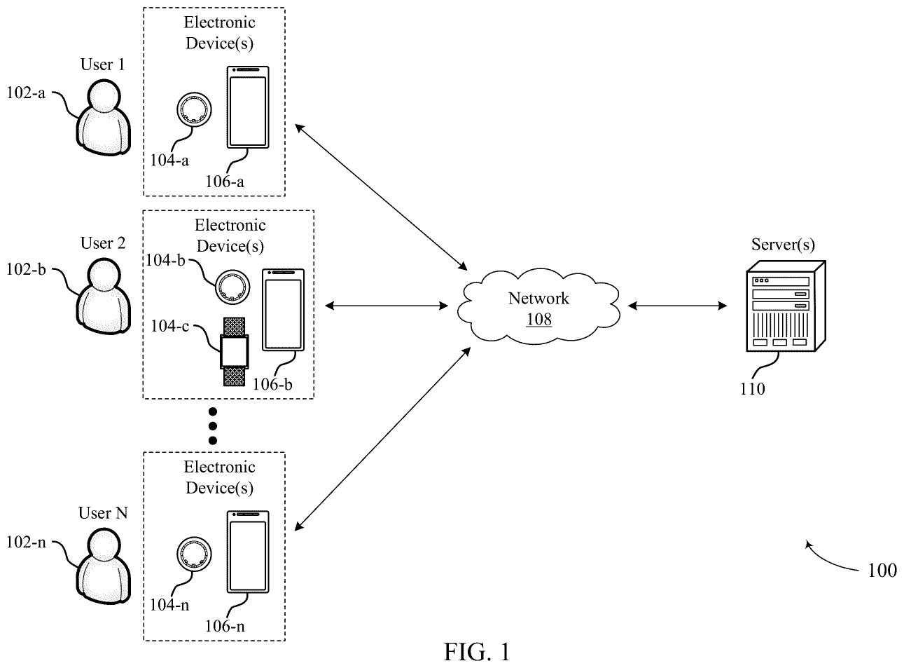

FIG. 1 illustrates an example of a system 100 that supports techniques for tactile and auditory respiration biofeedback in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IOT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state: 2) circadian rhythms: 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks”, 12 day rhythms could be used): 5) multi-day ovarian rhythms in women and spermatogenesis rhythms in men: 6) lunar rhythms (relevant for individuals living with low or no artificial lights): and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques that leverage physiological data, such as respiration data, collected via a ring 104, and vibrational capabilities, auditory capabilities, or both, of a user device 106 to support user relaxation (e.g., respiration awareness). In particular, aspects of the present disclosure may utilize respiration rate data (e.g., respiratory rate data, breathing rate data) collected during a respiration biofeedback session (e.g., relaxation session, biofeedback session, meditation session) to determine a respiration rate pattern in order to generate a set of tactile vibration pulses, a set of audio pulses, or both, to guide a user 102 to adjust (e.g., reduce) a respiration rate of the user 102. By reducing the respiration rate of the user 102, aspects of the present disclosure may aid in relaxation of the user 102, that may result in improved scores associated with the user 102 (e.g., Sleep Score, Readiness Score), improved physiological conditions of the user 102, improved psychological state of the user 102, or the like thereof.

For example, the system 100 may support a respiration biofeedback mode (e.g., relaxation mode, biofeedback mode) that enables a user 102-a to initiate a respiration biofeedback session via a user device 106-a. During the respiration biofeedback session, the system 100 may acquire physiological data associated with the user 102-a, the physiological data including at least respiration rate data. The system 100 may determine a first respiration rate associated with the user 102-a based on the respiration rate data, where the first respiration rate includes an inhalation rate (e.g., inhalation respiratory rate) of the user 102-a, an exhalation rate of the user 102-a (e.g., exhalation respiratory rate), or both.

In some examples, the system 100 may determine a respiration rate pattern based on the first respiration rate. That is, the respiration rate pattern may be an artificial respiration rate pattern that is different (e.g., lower, slower) than the first respiration rate of the user 102-a. Additionally, or alternatively, the system 100 may determine a respiration rate pattern based on a respiration rate of another user 102, a predefined respiration rate pattern, or the like thereof. For example, the system 100 may receive, via the network 108, respiration rate data associated with a user 102-b and may determine a respiration rate pattern based on the respiration rate data associated with the user 102-b. In another example, the system may receive, from a meditation application on the user device 106-a, an indication of a predefined respiration rate pattern associated with a meditation session.

Subsequently, the system 100 may generate, via the user device 106-a during the respiration biofeedback session, a set of tactile vibrational pulses (e.g., vibration pulses), a set of audio pulses (e.g., sound pulses), or both, in accordance with the respiration rate pattern (e.g., inhalation pattern and exhalation pattern). That is, the system 100 may instruct the user 102-a to inhale and exhale according to the respiration rate pattern via the set of tactile vibration pulses, the set of audio pulses, or both. For example, the system may instruct the user 102-a to place their hand on the user device 106-a and may generate the set of tactile vibration pulses according to the respiration rate pattern, instructing the user 102-a to breathe according to the respiration rate pattern generated via the set of tactile vibration pulses.

Additionally, or alternatively, the system 100 may generate the set of audio pulses according to the respiration rate pattern, instructing the user 102-a to breathe according to the respiration rate pattern generated via the set of audio pulses. In other words, the system may instruct the user 102-a to inhale during a first set of audio pulses generated according to the inhalation pattern (e.g., inhalation rate pattern) and to exhale during a second set of audio pulses generated according to the exhalation pattern (e.g., exhalation rate pattern). The audio pulses may be beeps, chimes, or any other form of sound pulse that may be generated in accordance with the respiration rate pattern. In additional or alternative implementations, the set of tactile vibrational pulses, the set of audio pulses, or both, may be generated via the wearable device 104.

In some examples, the system 100 may initiate the respiration biofeedback session based on a user input. That is, the user 102-a may manually initiate the respiration biofeedback session via the user device 106-a, the ring 104-a, or both. Additionally, or alternatively, the system 100 may prompt the user 102-a to initiate the respiration biofeedback session. In some examples, the system 100 may prompt the user 102-a to initiate a respiration biofeedback session based on a suggested bedtime associated with the user 102-a, a circadian rhythm associated with the user 102-a, prior respiration biofeedback sessions associated with the user 102-a (e.g., when the user 102-a has performed respiration biofeedback sessions in the past), additional physiological data associated with the user 102-a (e.g., heart rate data, respiration rate data, one or more stress indicator) or any combination thereof. Additionally, or alternatively, the system 100 may identify that the second user 102-b initiated a respiration biofeedback session and may prompt the first user 102-a to initiate a respiration biofeedback session (e.g., shared respiration biofeedback session) based on the second user 102-b. In other words, the system 100 associated with the first user 102-a may be connected to a system 100 associated with the second user 102-b via the network 108 (e.g., via any combination of the user device 106-a and/or the ring 104-a and the user device 106-b, the ring 104-b, and/or the watch 104-c) and may prompt the first user 102-a to initiate a respiration biofeedback session based on the second user 102-b initiating a respiration biofeedback session. In some examples, the respiration biofeedback session may be a shared (e.g., paired) respiration biofeedback session, such that a respiration biofeedback session associated with the first user 102-a is the same (e.g., in sync) as a respiration biofeedback session associated with the second user 102-b. A shared respiration biofeedback session may include any quantity of users 102.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

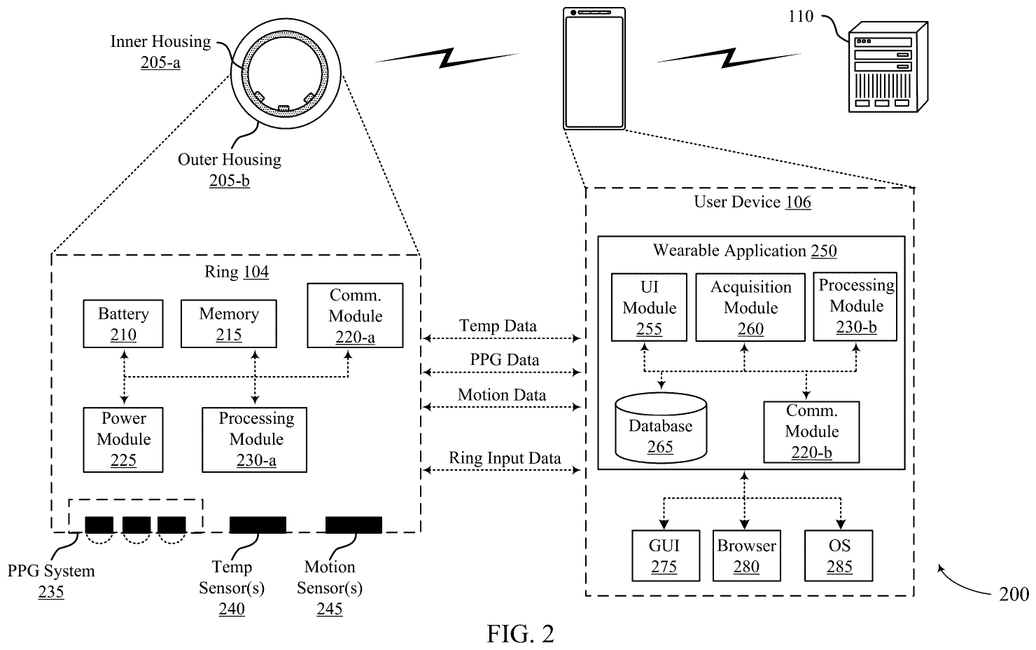

FIG. 2 illustrates an example of a system 200 that supports techniques for tactile and auditory respiration biofeedback in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamp, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-a. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, that may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate (e.g., respiration rate) based on frequency modulation, amplitude modulation, or baseline modulation of the user's IBI values over a period of time. Respiratory rate may be calculated in breaths per minute or as another respiration rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep day's may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency.” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° ° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the respective devices of the system 200 may support techniques that leverage physiological data, such as respiration data, collected via a ring 104, and vibrational capabilities, auditory capabilities, or both, of a user device 106 to support user relaxation. In particular, aspects of the present disclosure may utilize respiration rate data collected during a respiration biofeedback session (e.g., relaxation session) to determine a respiration rate pattern in order to generate a set of tactile vibration pulses, a set of audio pulses, or both, to guide a user 102 to adjust (e.g., reduce) a respiration rate of the user 102. By reducing the respiration rate of the user 102, aspects of the present disclosure may aid in relaxation of the user 102, that may result in improved scores associated with the user 102 (e.g., Sleep Score, Readiness Score), improved physiological conditions of the user 102, improved psychological state of the user 102, or the like thereof.

For example, the system 200 may support a respiration biofeedback mode that enables a user 102 to initiate a respiration biofeedback session via a user device 106 (e.g., via a GUI 275). During the respiration biofeedback session, the system 200 may acquire physiological data associated with the user 102, the physiological data including at least respiration rate data. The system may 200 determine a first respiration rate associated with the user 102 based on the respiration rate data, where the first respiration rate includes an inhalation rate of the user 102, an exhalation rate of the user 102, or both.

In some examples, the system 200 may determine a respiration rate pattern based on the first respiration rate. That is, the respiration rate pattern may be an artificial respiration rate pattern that is lower (e.g., slower) than the first respiration rate of the user 102. Additionally, or alternatively, the system 200 may determine a respiration rate pattern based on a respiration rate of another user 102, a predefined respiration rate pattern, or the like thereof. For example, the system 200 may receive respiration rate data associated with a second user 102 and may determine a respiration rate pattern based on the respiration rate data associated with the second user 102. In another example, the system may receive, from a meditation application on the user device 106, an indication of a predefined respiration rate pattern associated with a meditation session.

Subsequently, the system 200 may generate, via the user device 106 during the respiration biofeedback session, a set of tactile vibrational pulses (e.g., vibration pulses), a set of audio pulses (e.g., sound pulses), or both, in accordance with the respiration rate pattern (e.g., inhalation pattern and exhalation pattern). That is, the system 200 may instruct the user 102 to inhale and exhale according to the respiration rate pattern via the set of tactile vibration pulses, the set of audio pulses, or both. For example, the system may instruct the user 102 to place their hand on the user device 106 and may generate the set of tactile vibration pulses according to the respiration rate pattern, instructing the user 102 to breathe according to the respiration rate pattern generated via the set of tactile vibration pulses.

Additionally, or alternatively, the system 200 may generate the set of audio pulses according to the respiration rate pattern, instructing the user 102 to breathe according to the respiration rate pattern generated via the set of audio pulses. In other words, the system may instruct the user 102 to inhale during a first set of audio pulses generated according to the inhalation pattern and to exhale during a second set of audio pulses generated according to the exhalation pattern. The audio pulses may be beeps, chimes, or any other form of sound pulse that may be generated in accordance with the respiration rate pattern.

In some examples, the system 200 may adjust temperature data associated with the user 102 based on the user 102 initiating the respiration biofeedback mode. That is, the user 102 may place their hand on the user device 106 such that one or more temperature sensors 240 on the ring 104 may acquire temperature data associated with user 102 that is impacted (e.g., increased) by a temperature of the user device 106. Accordingly, the system 200 may determine the temperature of the user device 106 and may selectively modify the temperature data associated with the user 102 (e.g., collected via temperature sensors 240) based on the temperature of the user device 106 (e.g., and based on the hand of the user 102 being place on the user device 106). Stated differently, the system 200 may be configured to account for the temperature of the user device 106 throughout the respiration biofeedback session in order to determine more accurate temperature readings for the user.

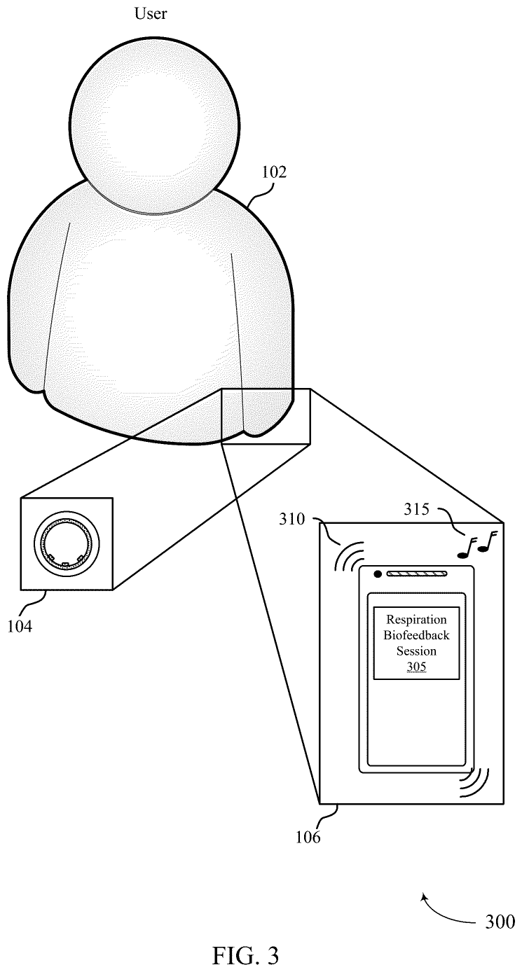

FIG. 3 illustrates an example of a system 300 that supports techniques for tactile and auditory respiration biofeedback in accordance with aspects of the present disclosure. The system 300 may implement, or be implemented by, aspects of the system 100, the system 200, or both.

In some aspects, the respective devices of the system 300 may support techniques that leverage physiological data, such as respiration data, collected via a ring 104, and vibrational capabilities (e.g., tactile, haptic), auditory capabilities, or both, of a user device 106 and/or the ring 104 to support user relaxation. In particular, aspects of the present disclosure may utilize respiration rate data collected during a respiration biofeedback session 305 to determine a respiration rate pattern in order to generate a set of tactile vibration pulses 310, a set of audio pulses 315, or both, to guide a user 102 to adjust (e.g., reduce) a respiration rate of the user 102. By reducing the respiration rate of the user 102, aspects of the present disclosure may aid in relaxation of the user 102, that may result in improved scores associated with the user 102 (e.g., Sleep Score, Readiness Score, Meditation Score, Relaxation Score), improved physiological conditions of the user 102, improved psychological state of the user 102, or the like thereof.

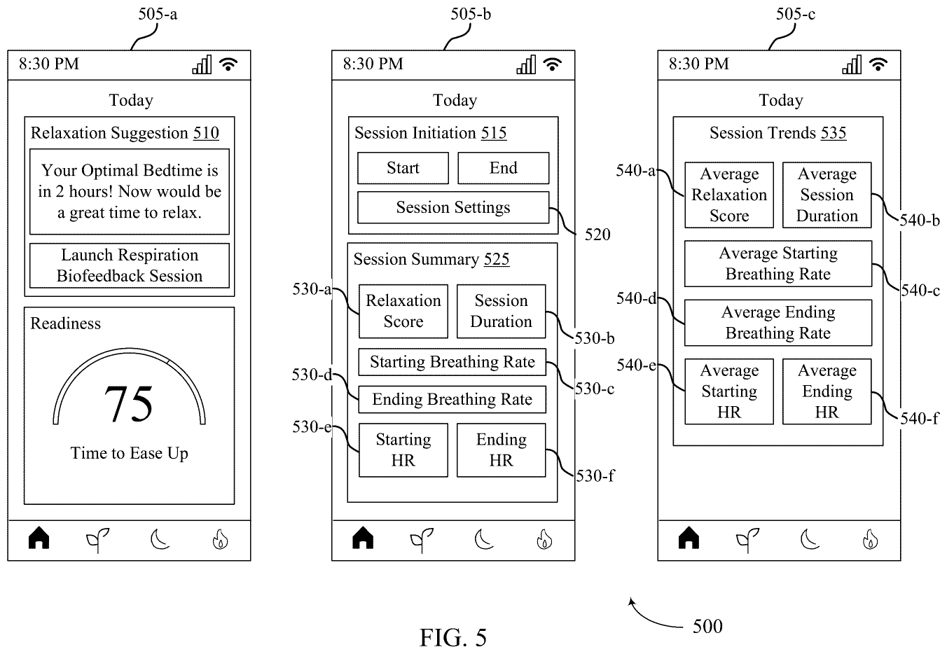

For example, the system 300 may support a respiration biofeedback mode that enables a user 102 to initiate a respiration biofeedback session 305 via a user device 106 (e.g., via a GUI 275). In some examples, the system 300 may initiate a respiration biofeedback session 305 based on a user input. That is, the user 102 may initiate a respiration biofeedback session 305 (e.g., without being prompted) via the user device 106 (e.g., via a GUI 500, as described with reference to FIG. 5), via the ring 104, or both. In some examples, the system 300 may prompt the user 102 to initiate the respiration biofeedback session 305 based on a bedtime associated with the user 102. For example, the system 300 may calculate a bedtime (e.g., suggested bedtime, optimal bedtime, calculated bedtime) associated with the user 102 based on a circadian rhythm associated with the user 102, based on previous bedtimes of the user 102, based on physiological data associated with the user 102, or the like thereof, and may prompt the user to initiate the respiration biofeedback session 305 based on the calculated bedtime. In other words, the system 300 may determine that the time of day is within a threshold time duration of the calculated bedtime and may prompt the user 102 (e.g., two hours prior to the calculated bedtime) to initiate the respiration biofeedback session 305 to aid in relaxing before bedtime, that may result in an improved Sleep Score.

Additionally, or alternatively, the system 300 may prompt the user to initiate the respiration biofeedback session 305 based on a circadian rhythm associated with the user 102. For example, the system 300 may determine, based on the circadian rhythm associated with the user 102, a time (e.g., optimal time) that a respiration biofeedback session 305 may result in improvements to one or more physiological conditions of the user 102, one or more psychological conditions of the user 102, or both. For example, the system 300 may determine that participating in a respiration biofeedback session 305 between 4:00 μm and 6:00 pm may result in improvements to how quickly the user 102 falls asleep (e.g., improving a Sleep Score of the user 102).

Additionally, or alternatively, the system 300 may prompt the user 102 to initiate a respiration biofeedback session 305 based on additional physiological data associated with the user 102, that may include, but is not limited to, a heart rate of the user 102, a respiration rate of the user 102, one or more stress indicators associated with user 102, or the like thereof. For example, the system 300 may determine that the user 102 is experiencing an elevated heart rate and an increased respiration rate but is not participating in physical activity (e.g., that may be a sign of stress or anxiety) and may prompt the user to initiate a respiration biofeedback session 305 to aid reducing the heart rate, the respiration rate, or both (e.g., aid in stress reduction).

Additionally, or alternatively, the system 100 may identify that another user 102 connected with the user 102, as described with reference to FIG. 2, initiated a respiration biofeedback session 305 and may prompt the user 102 to initiate a respiration biofeedback session 305 based on the other user 102. In other words, a system 300 associated with the user 102 may be connected to a system 300 associated with the other user 102 (e.g., a spouse, training partner, fitness/yoga instructor, etc.) and may prompt the user 102 to initiate a respiration biofeedback session 305 based on the other user 102 initiating a respiration biofeedback session 305. In some examples, the respiration biofeedback session 305 may be a shared (e.g., paired) respiration biofeedback session 305, such that a respiration biofeedback session 305 associated with the user 102 is the same (e.g., in sync) as a respiration biofeedback session 305 associated with the other user 102 (such as during a yoga or meditation class).

During the respiration biofeedback session 305, the system 300 may acquire physiological data associated with the user 102 via a ring 104, the physiological data including at least respiration rate data. The system 300 may determine a first respiration rate associated with the user 102 based on the respiration rate data, where the first respiration rate includes an inhalation rate (e.g., respiration rate during inhale) of the user 102, an exhalation rate (e.g., respiration rate during exhale) of the user 102, or both.

In some aspects, the wearable device 104 and/or user device 106 (e.g., processing module 230-a) may determine an inhalation respiratory rate (e.g., inhalation rate), an exhalation respiratory rate (e.g., exhalation rate), or both, based on frequency modulation, amplitude modulation, or baseline modulation of the user's IBI values over a period of time. For example, the system may calculate how many times per minute that the user inhales and/or exhales. Additionally, or alternatively, the system may calculate a duration associated with the user 102 inhaling, a duration associated with the user 102 exhaling, or both (e.g., how long the user takes to inhale/exhale). The wearable device 104 and/or user device 106 may store user respiratory rate values (e.g., and/or any data associated with respiration of the user 102) over time in memory. User respiratory rate values (e.g., user inhalation rate values, user exhalation rate values) and/or any other data associated with respiration of the user 102 (e.g., data associated with calculation of the user 102 respiratory rate values, duration of respiration, duration of inhale, duration of exhale, etc.) may be referred to as respiration rate data.

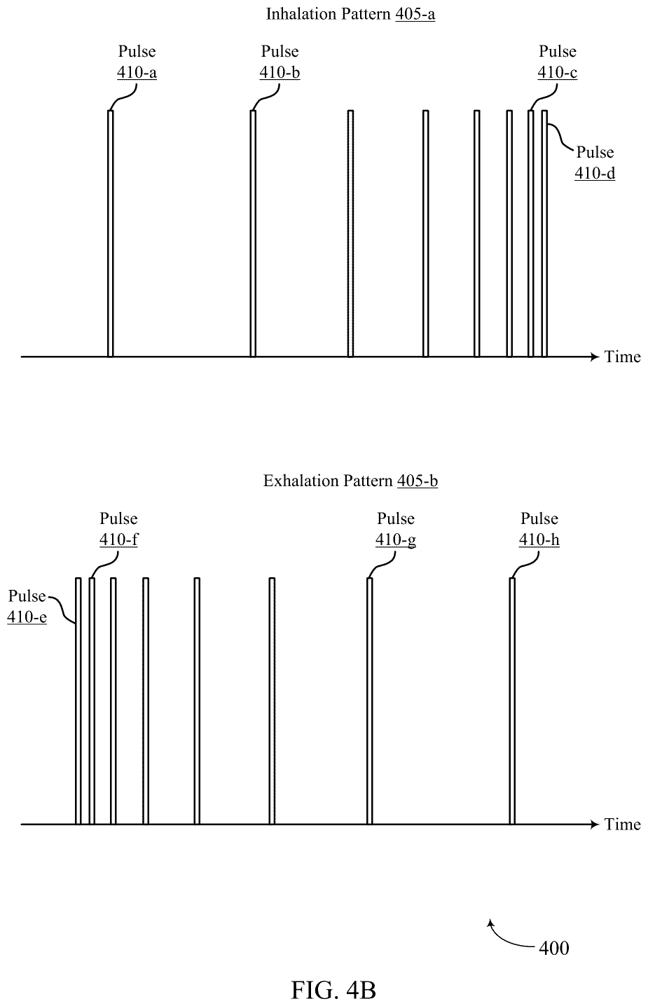

Additionally, the system 300 may determine a respiration rate pattern, described with reference to FIGS. 4A and 4B. In some examples, the respiration rate pattern may be based on the first respiration rate. That is, the respiration rate pattern may include an artificial respiration rate pattern that is lower (e.g., slower) than the first respiration rate of the user 102. In this regard, the artificial respiration rate pattern may include a pattern that is determined based on the user's current respiration rate, and which is configured to help assist the user 102 to lower their respiration rate to the lower respiration rate pattern. For instance, if the user's current respiration rate pattern is at 15 breaths per minute, the artificial respiration rate pattern may be determined at 12 or 13 breaths per minute to help the user 102 lower their respiration rate.

Additionally, or alternatively, the system 300 may determine a respiration rate pattern based on a respiration rate of another user 102, a predefined respiration rate pattern (e.g., a “target” respiration rate/pattern inputted by the user 102), or the like thereof. For example, the system 300 may receive respiration rate data associated with a second user 102 and may determine a respiration rate pattern based on the respiration rate data associated with the second user 102. In another example, the system may receive, from a meditation application on the user device 106, an indication of a predefined respiration rate pattern associated with a meditation session (e.g., further associated with the respiration biofeedback session).

Subsequently, the system 300 may generate a set of tactile vibration pulses 310 (e.g., vibration pulses), a set of audio pulses 315 (e.g., sound pulses), or both, using the user device 106 and/or wearable device 104 as part of the relaxation session and in accordance with the respiration rate pattern (e.g., inhalation pattern and exhalation pattern). That is, the system 300 may instruct the user 102 to inhale and exhale according to the respiration rate pattern via the set of tactile vibration pulses 310, the set of audio pulses 315, or both. For example, in cases where the set of tactile vibration pulses 310 are generated via the user device 106, the system 300 may instruct the user 102 to place their hand on the user device 106 and may generate the set of tactile vibration pulses 310 according to the respiration rate pattern, instructing the user 102 to breathe according to the respiration rate pattern generated via the set of tactile vibration pulses 310. In other cases, the set of tactile vibration pulses 310 may be generated via the wearable device 104. In some examples, the user 102 may enable, disable, or modify one or more settings associated with the set of tactile vibration pulses 310 via the user device 106, as described with reference to FIG. 5.

Additionally, or alternatively, the system 300 may generate the set of audio pulses 315 according to the respiration rate pattern, instructing the user 102 to breathe according to the respiration rate pattern generated via the set of audio pulses 315. In other words, the system 300 may instruct the user 102 to inhale during a first set of audio pulses 315 generated according to the inhalation pattern and to exhale during a second set of audio pulses 315 generated according to the exhalation pattern. The audio pulses 315 may be beeps, chimes, or any other form of sound pulse that may be generated in accordance with the respiration rate pattern. In some examples, the user 102 may enable, disable, or modify one or more settings associated with the set of audio pulses 315 via the user device 106, as described with reference to FIG. 5.

In some implementations, the set of tactile vibration pulses 310, the set of audio pulses 315, or both, may additionally or alternatively be generated by the wearable device 104. For example, in some cases, the wearable device 104 may generate the set of tactile vibration pulses 310 in accordance with the respiration rate pattern to help the user lower their respiration rate throughout the respiration biofeedback session 305.

Additionally, or alternatively, during the respiration biofeedback session 305, the system 300 may generate one or more audio cues. For example, the system 300 may instruct the user 102 to relax their posture, to shrug their shoulders, or to perform any other action that may aid in the relaxation of the user 102 (e.g., may aid in reducing a respiration rate of the user 102). Additionally, or alternatively, the one or more audio cues may include sounds, such as music, audiobooks, podcasts, white noise, brown noise, background noise, soundscapes, or the like thereof, to aid the relaxation of the user during the respiration biofeedback session 305. In some examples, the user 102 may enable, disable, or modify the one or more audio cues via the user device 106, as described with reference to FIG. 5.

As will be described in further detail herein, the system 300 may monitor the user's physiological data throughout (and/or after an end of) the respiration biofeedback session 305 to determine if/how the respiration biofeedback session 305 has affected the user's physiological data. For example, the wearable device 104 may collect temperature data, respiration rate data, heart rate data, and the like, throughout the respiration biofeedback session 305 to determine how the respiration biofeedback session 305 affected the respective physiological parameters.

In cases where the user 102 places their hand on the user device 106 to feel the set of tactile vibration pulses 310, the temperature from the user device 106 may affect temperature measurements (e.g., skin temperature measurements) acquired by the wearable device 104 (such as in cases where the wearable device 104 includes a ring, and where the user 102 places the hand including the ring on the user device 106). Accordingly, in some cases, the system 300 may be configured to determine a temperature of the user device 106, and may adjust temperature data collected by the wearable device 104 based on the temperature of the user device 106. Moreover, in some cases, the system 300 may determine whether the user's hand including the wearable ring device 104 is positioned on/around the user device 106 (e.g., based on signal strength measurements between the wearable ring device 104 and the user device 106) in order to determine if/when the system 300 needs to account for the temperature of the wearable device.

FIG. 4 illustrates an example of a respiration rate pattern 400 that supports techniques for tactile auditory respiration biofeedback in accordance with aspects of the present disclosure. The respiration rate pattern 400 (e.g., including the inhalation pattern 405-a and exhalation pattern 405-b) may implement, or be implemented by, aspects of the system 100, the system 200, the system 300, or any combination thereof.

As described previously with reference to FIG. 3, a system (e.g., such as the system 300) may determine a respiration rate pattern 400 associated with generation of a set of tactile vibration pulses, a set of audio pulses, or both, during a respiration biofeedback session. In the context of FIG. 4, the pulses 410 may represent vibration pulses, audio pulses, or both.

The respiration rate pattern 400 (e.g., determined by the system) may include an inhalation pattern 405-a and an exhalation pattern 405-b. In some cases, the inhalation pattern 405-a may include a set of pulses 410, including a pulse 410-a, a pulse 410-b, a pulse 410-c, and a pulse 410-d, associated with a decreasing frequency. That is, a first duration (e.g., time duration) between subsequent pulses 410 at the beginning of the inhalation pattern 405-a may be longer than a second duration between subsequent pulses 410 at the end of the inhalation pattern 405-a, such that a duration between subsequent pulses 410 decreases as the inhalation pattern 405-a progresses (e.g., with respect to time). For example, a first time duration between the pulse 410-a and the pulse 410-b may be greater than a second time duration between the pulse 410-c and the pulse 410-d.

Conversely, the exhalation pattern 405-b may include a set of pulses 410, including a pulse 410-e, a pulse 410-f, a pulse 410-g, and a pulse 410-h, associated with an increasing frequency. That is, a first duration (e.g., time duration) between subsequent pulses 410 at the beginning of the exhalation pattern 405-b may be shorter than a second duration between subsequent pulses 410 at the end of the exhalation pattern 405-b, such that a duration between subsequent pulses 410 increases as the exhalation pattern 405-b progresses (e.g., with respect to time). For example, a first time duration between the pulse 410-e and the pulse 410-f may be greater than a second time duration between the pulse 410-g and the pulse 410-h.

The changing frequencies of pulses 410 in the inhalation pattern 405-a and the exhalation pattern 405-b may assist the user in slowing their respiration rate. Moreover, a user may determine when to transition from inhalation to exhalation (e.g., and visa-versa) based on changes in the frequency of pulses 410. In other words, the user may inhale during a time duration associated with the inhalation pattern 405-a and may transition to exhaling for a second time duration associated with the exhalation pattern 405-b based on a frequency of pulses 410 transitioning from an increasing frequency to a decreasing frequency. In some examples, the first time duration and the second time duration may be the same. Additionally, or alternatively, a rate that the frequency of pulses 410 increases with respect to the inhalation pattern 405-a may be the same as a rate that the frequency of pulses 410 decreases with respect to the exhalation pattern 405-b.

In some examples, the system may determine the respiration rate pattern 400 based on a respiration rate associated with the user. That is, the system may acquire physiological data, including respiration rate data, associated with the user and may determine a first respiration rate associated with the user. Additionally, the system may determine a second (e.g., artificial) respiration rate based on the first respiration rate, such that the second respiration rate is lower (e.g., slower) than the first respiration rate. The system may determine the respiration rate pattern 400 based on the second respiration rate. That is, the system may determine the first duration associated with the inhalation pattern 405-a and the second duration associated with the exhalation pattern 405-b based on the second respiration rate.

Additionally, or alternatively, the system may determine the respiration rate pattern 400 based on a second user associated with the user, that may be referred to as a first user in the context of this example. That is, the system associated with the first user, that may be referred to as a first system in the context of this example, may be associated with a second system further associated with the second user. That is, the second system may share data (e.g., physiological data, tags, insights, scores, etc.) associated with the second user with the first system (e.g., and visa-versa). In other words, the second system may share respiration rate data, a respiration rate, a respiration rate pattern 400, or any combination thereof, associated with the second user with the first system. As such, the first system may determine a respiration rate pattern 400 for the first user based on the respiration rate data, the respiration rate, the respiration rate pattern 400, or any combination thereof, associated with the second user. For example, the second user may initiate a respiration biofeedback session via the second system and the first user may initiate a respiration biofeedback session via the first system (e.g., based on the second user initiating the respiration biofeedback session) such that the first user and the second user may participate in a shared respiration biofeedback session. In such cases, the first system, the second system, or both, may determine a shared respiration rate pattern 400 based on a respiration rate of the first user, a respiration rate of the second user, or both.

Additionally, or alternatively, the system may determine the respiration rate pattern 400 based on a predefined respiration rate pattern 400. That is, the system may communication with one or more applications (e.g., during a respiration biofeedback session), such as a meditation app, a respiration biofeedback app, or the like thereof, and may receive (e.g., from the one or more applications) an indication of a respiration rate, a respiration rate pattern 400, or both, such that the system may determine a respiration rate pattern 400 for the user based on the indicated respiration rate, the indicated respiration rate pattern 400, or both. Additionally, or alternatively, the user may be able to input a “target” or “goal” respiration rate, where the respiration rate pattern 400 is determined based on the inputted target/goal respiration rate.

Additionally, or alternatively, the system may determine the respiration rate pattern 400 based on additional physiological data associated with the user. For example, the system may determine a heart rate of the user and determine the respiration rate pattern 400 such that the pulses 410 align with (e.g., are at the same time as) a heart rate rhythm of the user.

In some examples, the system may incrementally reduce a respiration rate of the user based on a difference between the first respiration rate and a desired respiration rate. That is, the difference between the first respiration rate and the desired respiration rate may exceed a threshold such that the system may determine a set of respiration rate patterns 400. In such cases, the system may generate pulses 410 according to a first respiration rate pattern 400 from the set of respiration rate patterns 400 and may switch to generating pulses 410 according to second respiration rate pattern 400 from the set of respiration rate patterns 400 based on a respiration rate of the user being within a threshold (e.g., being the same as) of a respiration rate associated with the first respiration rate pattern. That is, each respiration rate pattern 400 of the set of respiration rate patterns may be associated with a respiration rate such that a first respiration rate associated with a first respiration rate pattern 400 of the set of respiration rate patterns may be higher (e.g., faster) than a final (e.g., last) respiration rate (e.g., desired respiration rate) associated with a final respiration rate pattern 400 of the set of respiration rate patterns 400. In such cases, each respiration rate associated with subsequent respiration rate patterns 400 may be lower (e.g., slower) than a previous respiration rate associated with a previous respiration rate pattern 400. In some examples, the rate that respective respiration rates associated with the set of respiration rate patterns 400 decreases may be based on the difference between the respiration rate of the user and the desired respiration rate (e.g., associated with the final respiration rate pattern 400).

In some cases, physiological data (e.g., respiration rate data, heart rate data, HRV data, acceleration/motion data, bioimpedance data, etc.) collected during the respiration biofeedback session 305 may be used to selectively adjust one or more parameters of the set of the respiration rate pattern 400. Parameters of the respiration rate pattern 400 that may be adjusted may include, but are not limited to, a magnitude/intensity of the set of tactile vibration pulses 410, a volume of the set of audio pulses 410, a duration of each tactile vibration/audio pulse 410, a frequency of the pulses 410, or any combination thereof. For example, the system 300 may adjust a magnitude of tactile vibration pulses 410 based on temperature and/or motion data collected via the wearable device 104 to ensure that the user is able to feel the tactile vibration pulses 410.

FIG. 5 illustrates an example of a GUI 500 that supports techniques for tactile and auditory respiration biofeedback in accordance with aspects of the present disclosure. The GUI 500 may implement, or be implemented by, aspects of the system 100, the system 200, the system 300, the respiration rate pattern 400, or any combination thereof.

The GUI 500 illustrates a series of application pages 505 that may be displayed to the user via the GUI 500 (e.g., GUI 275 illustrated in FIG. 2). Continuing with the example in FIG. 3, the system 300 may prompt the user 102 to initiate a respiration biofeedback session (e.g., relaxation session) such that the user 102 may be presented with the application page 505-a, displaying a relaxation suggestion 510 (e.g., respiration biofeedback suggestion 510). In some examples, the relaxation suggestion 510 may display an indication of a reason associated with the relaxation suggestion 510. For example, the relaxation suggestion 510 may indicate that the user 102 is within 2 hours of an optimal bedtime and performing a respiration biofeedback session may improve sleep of the user 102 (e.g., improve a Sleep Score associated with the user 102). Additionally, or alternatively, the relaxation suggestion 510 may enable the user 102 to launch a respiration biofeedback session. In some cases, the application page 505-a may display the one or more scores for the user for the respective day.

In cases where the user 102 dismisses (e.g., denies) the relaxation suggestion 510 (.g., prompt) on the application page 505-a, the relaxation suggestion 510 may disappear. Additionally, or alternatively, the user 102 may be presented with the application page 505-b. As shown in FIG. 5, the application page 505-b may display a session initiation 515 prompting the user to initiate (e.g., start) and/or stop a respiration biofeedback session. In some examples, the system 300 may stop the respiration biofeedback session after a predetermined quantity of time, after a user input stopping the respiration biofeedback session (e.g., via the session initiation 515), or the like thereof.

In some examples, the session initiation 515 may prompt the user 102 to modify one or more session settings 520 associated with the respiration biofeedback session. In some examples, the respiration biofeedback session may be associated with a set of default session settings 520 (e.g., based on system 300 configuration, user selection, or both). Additionally, or alternatively, the user 102 may select one or more session settings 520 for a respiration biofeedback session. For example, the one or more session settings 520 may include a duration of the respiration biofeedback session, a set of settings associated with tactile vibration pulses, a set of settings associated with audio pulses, a set of settings associated with audio cues, or any combination thereof. The set of settings associated with the tactile vibration pulses may include an option for the user 102 to enable or disable the tactile vibration pulses (e.g., on the ring 104, the user device 106, or both), an intensity of vibration association with the tactile vibration pulses, a vibration pattern associated with the tactile vibration pulses, or the like thereof. The set of settings associated with the audio pulses may include an option for the user 102 to enable or disable the audio pulses, a volume of the audio pulses, a sound of the audio pulses, an audio pattern associated with the audio pulses, or the like thereof. The set of settings associated with the audio cues may include an option for the user 102 to enable or disable the audio cues, a type of audio cue (e.g., music, instructive cues, etc.), one or more settings associated with the type of audio cue (e.g., what music to play, what instructive cues to give, etc.), or the like thereof.

Additionally, upon completion of a respiration biofeedback session, the application page 505-b may display a session summary 525 (e.g., relaxation session summary 525, respiration biofeedback session summary 525). The session summary 525 may include one or more insights 530 associated with the respiration biofeedback session. For example, the one or more insights 530 may include, but is not limited to, an insight 530-a indicating a Relaxation Score (e.g., Respiration Biofeedback Score), an insight 530-b indicating a session duration, an insight 530-c indicating a breathing rate (e.g., respiration rate) of the user 102 at the beginning of the respiration biofeedback session (e.g., starting breathing rate), an insight 530-d indicating a breathing rate (e.g., respiration rate) of the user 102 at the end of the respiration biofeedback session (e.g., ending breathing rate), an insight 530-e indicating a heart rate of the user 102 at the beginning of the respiration biofeedback session (e.g., starting HR), an insight 530-f indicating a heart rate of the user 102 at the end of the respiration biofeedback session (e.g., ending HR), or any combination thereof. The insights 530 may include any indication of data associated with the user 102 (e.g., associated with the respiration biofeedback session), the respiration biofeedback session, or both. The Relaxation Score may be based on physiological data associated with the user 102 during the respiration biofeedback session. For example, the physiological data may include a heart rate, a heart rate variability (HRV), a finger temperature, a perfusion index, or any combination thereof.

In some examples, as shown in FIG. 5, the user may access an application page 505-c indicating session trends 535 (e.g., relaxation session trends 535, respiration biofeedback session trends 535). That is, the session trends 535 may display an indication of one or more trends 540 (e.g., trend data) associated with multiple respiration biofeedback sessions performed by the user 102. For example, the one or more trends 540 may include, but is not limited to, a trend 540-a indicating an average Relaxation Score (and/or average Meditation Score), a trend 540-b indicating an average session duration, a trend 540-c indicating an average breathing rate (e.g., respiration rate) of the user 102 at the beginning of the respiration biofeedback session (e.g., starting breathing rate), a trend 540-d indicating an average breathing rate (e.g., respiration rate) of the user 102 at the end of the respiration biofeedback session (e.g., ending breathing rate), a trend 540-e indicating an average heart rate of the user 102 at the beginning of the respiration biofeedback session (e.g., starting HR), a trend 540-f indicating an average heart rate of the user 102 at the end of the respiration biofeedback session (e.g., ending HR), or any combination thereof. Additionally, or alternatively, the one or more trends 540 may include a frequency that the user 102 performs respiration biofeedback sessions at different times of day, an indication of how performing respiration biofeedback sessions has impacted one or more scores (e.g., Sleep Score, Readiness Score, Meditation Score) of the user 102, how many respiration biofeedback sessions the user 102 has participated in, how many shared respiration biofeedback sessions the user 102 has participated in, or the like thereof. The trends 540 may include any indication of data associated with the user 102 (e.g., associated with the set of respiration biofeedback sessions), the set of respiration biofeedback sessions, or both.

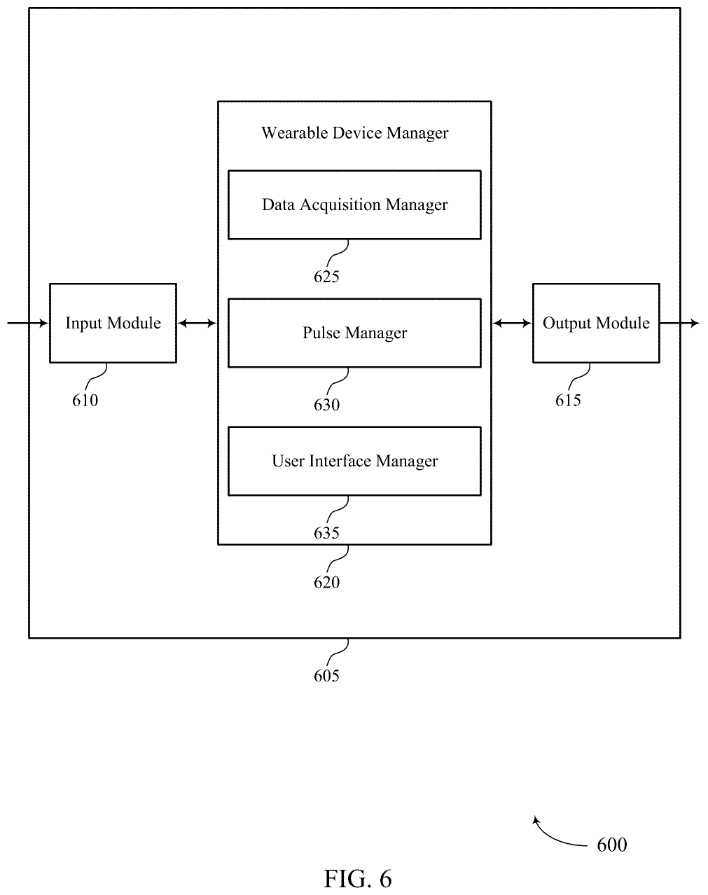

FIG. 6 illustrates a block diagram 600 of a device 605 that supports techniques for tactile and auditory respiration biofeedback in accordance with aspects of the present disclosure. The device 605 may include an input module 610, an output module 615, and a wearable device manager 620. The device 605 may also include a processor. Each of these components may be in communication with one another (e.g., via one or more buses).

For example, the wearable device manager 620 may include a data acquisition manager 625, a pulse manager 630, a user interface manager 635, or any combination thereof. In some examples, the wearable device manager 620, or various components thereof, may be configured to perform various operations (e.g., receiving, monitoring, transmitting) using or otherwise in cooperation with the input module 610, the output module 615, or both. For example, the wearable device manager 620 may receive information from the input module 610, send information to the output module 615, or be integrated in combination with the input module 610, the output module 615, or both to receive information, transmit information, or perform various other operations as described herein.

The data acquisition manager 625 may be configured as or otherwise support a means for acquiring physiological data associated with a user via a wearable device during a respiration biofeedback session, the physiological data comprising at least respiration rate data. The pulse manager 630 may be configured as or otherwise support a means for generating, via a user device during the respiration biofeedback session, a set of tactile vibration pulses, a set of audio pulses, or both, wherein the set of tactile vibration pulses, the set of audio pulses, or both, are generated in accordance with a respiration rate pattern that is based at least in part on the respiration rate data. The user interface manager 635 may be configured as or otherwise support a means for causing a graphical user interface of the user device to display an indication of one or more changes in the physiological data acquired during the respiration biofeedback session.

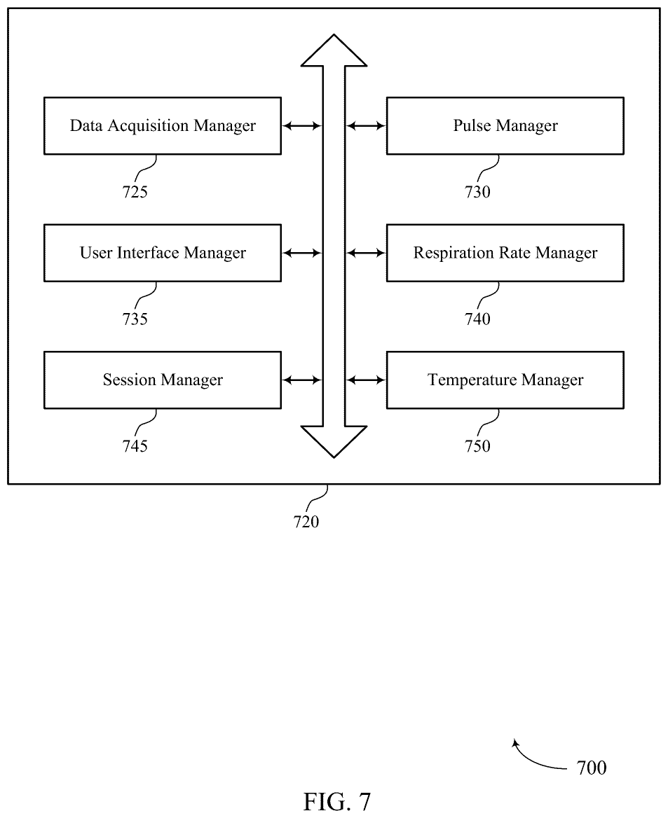

FIG. 7 illustrates a block diagram 700 of a wearable device manager 720 that supports techniques for tactile and auditory respiration biofeedback in accordance with aspects of the present disclosure. The wearable device manager 720 may be an example of aspects of a wearable device manager or a wearable device manager 620, or both, as described herein. The wearable device manager 720, or various components thereof, may be an example of means for performing various aspects of tactile and auditory respiration biofeedback as described herein. For example, the wearable device manager 720 may include a data acquisition manager 725, a pulse manager 730, a user interface manager 735, a respiration rate manager 740, a session manager 745, a temperature manager 750, or any combination thereof. Each of these components may communicate, directly or indirectly, with one another (e.g., via one or more buses).

The data acquisition manager 725 may be configured as or otherwise support a means for acquiring physiological data associated with a user via a wearable device during a respiration biofeedback session, the physiological data comprising at least respiration rate data. The pulse manager 730 may be configured as or otherwise support a means for generating, via a user device during the respiration biofeedback session, a set of tactile vibration pulses, a set of audio pulses, or both, wherein the set of tactile vibration pulses, the set of audio pulses, or both, are generated in accordance with a respiration rate pattern that is based at least in part on the respiration rate data. The user interface manager 735 may be configured as or otherwise support a means for causing a graphical user interface of the user device to display an indication of one or more changes in the physiological data acquired during the respiration biofeedback session.

In some examples, the respiration rate manager 740 may be configured as or otherwise support a means for determining a first respiration rate associated with the user based at least in part on the respiration rate data, wherein the respiration rate pattern is associated with an artificial respiration rate that is lower than the first respiration rate.

In some examples, the respiration rate pattern comprises an exhalation pattern and an inhalation pattern.

In some examples, the exhalation pattern is associated with a decelerating frequency for the set of tactile vibration pulses, the set of audio pulses, or both. In some examples, the inhalation pattern is associated with an increasing frequency for the set of tactile vibration pulses, the set of audio pulses, or both.

In some examples, the data acquisition manager 725 may be configured as or otherwise support a means for acquiring additional physiological data associated with the user via the wearable device during the respiration biofeedback session, the additional physiological data including at least additional respiration rate data. In some examples, the pulse manager 730 may be configured as or otherwise support a means for generating the set of tactile vibration pulses, the set of audio pulses, or both, in accordance with a second respiration rate pattern that is based at least in part on the additional respiration rate data.

In some examples, the user interface manager 735 may be configured as or otherwise support a means for causing the graphical user interface of the user device to display a prompt for the respiration biofeedback session based at least in part on a calculated bedtime associated with the user, a circadian rhythm associated with the user, prior respiration biofeedback sessions associated with the user, or any combination thereof, wherein acquiring the physiological data during the respiration biofeedback session is based at least in part on the prompt.

In some examples, the user interface manager 735 may be configured as or otherwise support a means for receiving, via the user device, the wearable device, or both, a user input initiating the respiration biofeedback session, wherein the user input is received in response to the prompt, and wherein acquiring the physiological data, generating the set of tactile vibration pulses, the set of audio pulses, or both, is based at least in part on the user input.

In some examples, the user interface manager 735 may be configured as or otherwise support a means for causing the graphical user interface of the user device to display a message instructing the user to place their hand on the user device based at least in part on receiving the user input, wherein generating the set of tactile vibration pulses is based at least in part on displaying the message.

In some examples, the session manager 745 may be configured as or otherwise support a means for initiating the respiration biofeedback session based at least in part on additional physiological data acquired from the user, the additional physiological data comprising a heart rate, a respiration rate, one or more stress indicators, and the like, wherein acquiring the physiological data is based at least in part on initiating the respiration biofeedback session.

In some examples, the physiological data comprises temperature data, heart rate data, heart rate variability data, acceleration data, bioimpedance data, or any combination thereof. In such examples, the respiration rate manager 740 may be configured as or otherwise support a means for determining one or more parameters of the respiration rate pattern based at least in part on the temperature data, the heart rate data, the heart rate variability data, the acceleration data, bioimpedance data, or any combination thereof.

In some examples, the one or more parameters comprise a magnitude of the set of tactile vibration pulses, a volume of the set of audio pulses, a duration of each tactile vibration pulse of the set of tactile vibration pulses, a duration of each audio pulse of the set of audio pulses, a frequency of the set of tactile vibration pulses, a frequency of the set of audio pulses, or any combination thereof.

In some examples, the physiological data further comprises temperature data and the wearable device comprises a wearable ring device worn on a finger of a hand of the user. In such cases, the temperature manager 750 may be configured as or otherwise support a means for determining a temperature of the user device based at least in part on determining that the hand of the user is placed on the user device during the respiration biofeedback session. In some examples, the temperature manager 750 may be configured as or otherwise support a means for selectively modifying the temperature data associated with the user based at least in part on the temperature of the user device and based at least in part on determining that the hand of the user is placed on the user device during the respiration biofeedback session.

In some examples, the wearable device comprises a wearable ring device.

In some examples, the wearable device collects the physiological data from the user based on arterial blood flow.

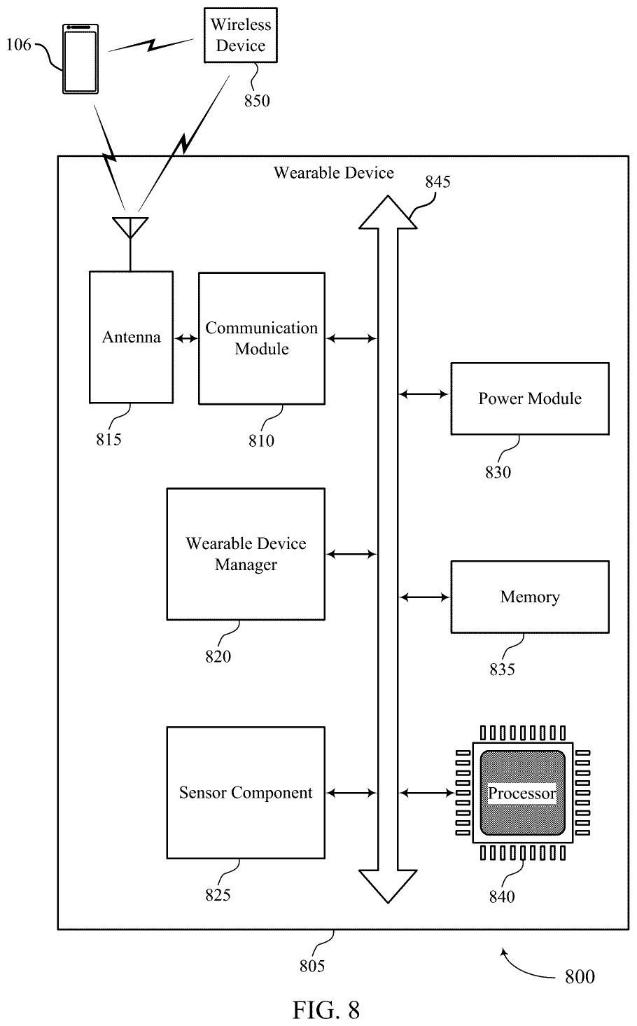

FIG. 8 illustrates a diagram of a system 800 including a device 805 that supports techniques for tactile and auditory respiration biofeedback in accordance with aspects of the present disclosure. The device 805 may be an example of or include the components of a device 605 as described herein. The device 805 may include an example of a wearable device 104, as described previously herein. The device 805 may include components for bi-directional communications including components for transmitting and receiving communications with a user device 106 and a server 110, such as a wearable device manager 820, a communication module 810, an antenna 815, a sensor component 825, a power module 830, a memory 835, a processor 840, and a wireless device 850. These components may be in electronic communication or otherwise coupled (e.g., operatively, communicatively, functionally, electronically, electrically) via one or more buses (e.g., a bus 845).

For example, the wearable device manager 820 may be configured as or otherwise support a means for acquiring physiological data associated with a user via a wearable device during a respiration biofeedback session, the physiological data comprising at least respiration rate data. The wearable device manager 820 may be configured as or otherwise support a means for generating, via a user device during the respiration biofeedback session, a set of tactile vibration pulses, a set of audio pulses, or both, wherein the set of tactile vibration pulses, the set of audio pulses, or both, are generated in accordance with a respiration rate pattern that is based at least in part on the respiration rate data. The wearable device manager 820 may be configured as or otherwise support a means for causing a graphical user interface of the user device to display an indication of one or more changes in the physiological data acquired during the respiration biofeedback session.

By including or configuring the wearable device manager 820 in accordance with examples as described herein, the device 805 may support techniques for tactile and auditory respiration biofeedback that may result in improved scores associated with a user (e.g., Sleep Score, Readiness Score), reduced stress levels of the user, reduced respiration rate of the user, or the like thereof.

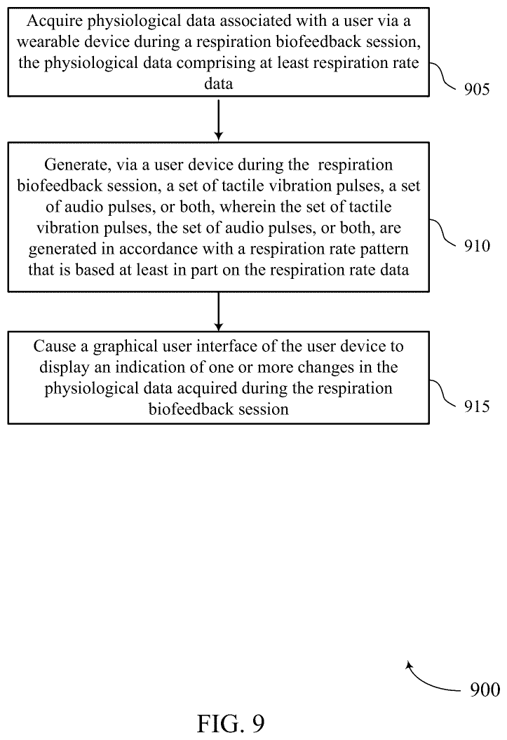

FIG. 9 illustrates a flowchart showing a method 900 that supports techniques for tactile and auditory respiration biofeedback in accordance with aspects of the present disclosure. The operations of the method 900 may be implemented by a wearable device or its components as described herein. For example, the operations of the method 900 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 905, the method may include acquiring physiological data associated with a user via a wearable device during a respiration biofeedback session, the physiological data comprising at least respiration rate data. The operations of 905 may be performed in accordance with examples as disclosed herein. In some examples, aspects of the operations of 905 may be performed by a data acquisition manager 725 as described with reference to FIG. 7.

At 910, the method may include generating, via a user device during the respiration biofeedback session, a set of tactile vibration pulses, a set of audio pulses, or both, wherein the set of tactile vibration pulses, the set of audio pulses, or both, are generated in accordance with a respiration rate pattern that is based at least in part on the respiration rate data. The operations of 910 may be performed in accordance with examples as disclosed herein. In some examples, aspects of the operations of 910 may be performed by a pulse manager 730 as described with reference to FIG. 7.

At 915, the method may include causing a graphical user interface of the user device to display an indication of one or more changes in the physiological data acquired during the respiration biofeedback session. The operations of 915 may be performed in accordance with examples as disclosed herein. In some examples, aspects of the operations of 915 may be performed by a user interface manager 735 as described with reference to FIG. 7.

It should be noted that the methods described above describe possible implementations, and that the operations and the steps may be rearranged or otherwise modified and that other implementations are possible. Furthermore, aspects from two or more of the methods may be combined.

A method is described. The method may include acquiring physiological data associated with a user via a wearable device during a respiration biofeedback session, the physiological data comprising at least respiration rate data, generating, via a user device during the respiration biofeedback session, a set of tactile vibration pulses, a set of audio pulses, or both, wherein the set of tactile vibration pulses, the set of audio pulses, or both, are generated in accordance with a respiration rate pattern that is based at least in part on the respiration rate data, and causing a graphical user interface of the user device to display an indication of one or more changes in the physiological data acquired during the respiration biofeedback session.

An apparatus is described. The apparatus may include a processor, memory coupled with the processor, and instructions stored in the memory. The instructions may be executable by the processor to cause the apparatus to acquire physiological data associated with a user via a wearable device during a respiration biofeedback session, the physiological data comprising at least respiration rate data, generate, via a user device during the respiration biofeedback session, a set of tactile vibration pulses, a set of audio pulses, or both, wherein the set of tactile vibration pulses, the set of audio pulses, or both, are generated in accordance with a respiration rate pattern that is based at least in part on the respiration rate data, and cause a graphical user interface of the user device to display an indication of one or more changes in the physiological data acquired during the respiration biofeedback session.

Another apparatus is described. The apparatus may include means for acquiring physiological data associated with a user via a wearable device during a respiration biofeedback session, the physiological data comprising at least respiration rate data, means for generating, via a user device during the respiration biofeedback session, a set of tactile vibration pulses, a set of audio pulses, or both, wherein the set of tactile vibration pulses, the set of audio pulses, or both, are generated in accordance with a respiration rate pattern that is based at least in part on the respiration rate data, and means for causing a graphical user interface of the user device to display an indication of one or more changes in the physiological data acquired during the respiration biofeedback session.

A non-transitory computer-readable medium storing code is described. The code may include instructions executable by a processor to acquire physiological data associated with a user via a wearable device during a respiration biofeedback session, the physiological data comprising at least respiration rate data, generate, via a user device during the respiration biofeedback session, a set of tactile vibration pulses, a set of audio pulses, or both, wherein the set of tactile vibration pulses, the set of audio pulses, or both, are generated in accordance with a respiration rate pattern that is based at least in part on the respiration rate data, and cause a graphical user interface of the user device to display an indication of one or more changes in the physiological data acquired during the respiration biofeedback session.

Some examples of the method, apparatuses, and non-transitory computer-readable medium described herein may further include operations, features, means, or instructions for determining a first respiration rate associated with the user based at least in part on the respiration rate data, wherein the respiration rate pattern may be associated with an artificial respiration rate that may be lower than the first respiration rate.

In some examples of the method, apparatuses, and non-transitory computer-readable medium described herein, the respiration rate pattern comprises an exhalation pattern and an inhalation pattern.

In some examples of the method, apparatuses, and non-transitory computer-readable medium described herein, the exhalation pattern may be associated with a decelerating frequency for the set of tactile vibration pulses, the set of audio pulses, or both and the inhalation pattern may be associated with an increasing frequency for the set of tactile vibration pulses, the set of audio pulses, or both.

Some examples of the method, apparatuses, and non-transitory computer-readable medium described herein may further include operations, features, means, or instructions for acquiring additional physiological data associated with the user via the wearable device during the respiration biofeedback session, the additional physiological data including at least additional respiration rate data and generating the set of tactile vibration pulses, the set of audio pulses, or both, in accordance with a second respiration rate pattern that may be based at least in part on the additional respiration rate data.

Some examples of the method, apparatuses, and non-transitory computer-readable medium described herein may further include operations, features, means, or instructions for causing the graphical user interface of the user device to display a prompt for the respiration biofeedback session based at least in part on a calculated bedtime associated with the user, a circadian rhythm associated with the user, prior respiration biofeedback sessions associated with the user, or any combination thereof, wherein acquiring the physiological data during the respiration biofeedback session may be based at least in part on the prompt.

Some examples of the method, apparatuses, and non-transitory computer-readable medium described herein may further include operations, features, means, or instructions for receiving, via the user device, the wearable device, or both, a user input initiating the respiration biofeedback session, wherein the user input may be received in response to the prompt, and wherein acquiring the physiological data, generating the set of tactile vibration pulses, the set of audio pulses, or both, may be based at least in part on the user input.

Some examples of the method, apparatuses, and non-transitory computer-readable medium described herein may further include operations, features, means, or instructions for causing the graphical user interface of the user device to display a message instructing the user to place their hand on the user device based at least in part on receiving the user input, wherein generating the set of tactile vibration pulses may be based at least in part on displaying the message.

Some examples of the method, apparatuses, and non-transitory computer-readable medium described herein may further include operations, features, means, or instructions for initiating the respiration biofeedback session based at least in part on additional physiological data acquired from the user, the additional physiological data comprising a heart rate, a respiration rate, one or more stress indicators, and the like, wherein acquiring the physiological data may be based at least in part on initiating the respiration biofeedback session.

In some examples of the method, apparatuses, and non-transitory computer-readable medium described herein, and the method, apparatuses, and non-transitory computer-readable medium may include further operations, features, means, or instructions for determining one or more parameters of the respiration rate pattern based at least in part on the temperature data, the heart rate data, the heart rate variability data, the acceleration data, bioimpedance data, or any combination thereof.

In some examples of the method, apparatuses, and non-transitory computer-readable medium described herein, the one or more parameters comprise a magnitude of the set of tactile vibration pulses, a volume of the set of audio pulses, a duration of each tactile vibration pulse of the set of tactile vibration pulses, a duration of each audio pulse of the set of audio pulses, a frequency of the set of tactile vibration pulses, a frequency of the set of audio pulses, or any combination thereof.

In some examples of the method, apparatuses, and non-transitory computer-readable medium described herein, and the method, apparatuses, and non-transitory computer-readable medium may include further operations, features, means, or instructions for determining a temperature of the user device based at least in part on determining that the hand of the user may be placed on the user device during the respiration biofeedback session and selectively modifying the temperature data associated with the user based at least in part on the temperature of the user device and based at least in part on determining that the hand of the user may be placed on the user device during the respiration biofeedback session.

In some examples of the method, apparatuses, and non-transitory computer-readable medium described herein, the wearable device comprises a wearable ring device.

In some examples of the method, apparatuses, and non-transitory computer-readable medium described herein, the wearable device collects the physiological data from the user based on arterial blood flow.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method comprising:
acquiring physiological data associated with a user via a wearable device during a respiration biofeedback session, the physiological data comprising at least respiration rate data:
generating, via a user device during the respiration biofeedback session, a set of tactile vibration pulses, a set of audio pulses, or both, wherein the set of tactile vibration pulses, the set of audio pulses, or both, are generated in accordance with a respiration rate pattern that is based at least in part on the respiration rate data; and
causing a graphical user interface of the user device to display an indication of one or more changes in the physiological data acquired during the respiration biofeedback session.

2. The method of claim 1, further comprising:
determining a first respiration rate associated with the user based at least in part on the respiration rate data, wherein the respiration rate pattern is associated with an artificial respiration rate that is lower than the first respiration rate.

3. The method of claim 1, wherein the respiration rate pattern comprises an exhalation pattern and an inhalation pattern.

4. The method of claim 3, wherein the exhalation pattern is associated with a decelerating frequency for the set of tactile vibration pulses, the set of audio pulses, or both, and wherein the inhalation pattern is associated with an increasing frequency for the set of tactile vibration pulses, the set of audio pulses, or both.

5. The method of claim 1, further comprising:
acquiring additional physiological data associated with the user via the wearable device during the respiration biofeedback session, the additional physiological data comprising at least additional respiration rate data; and
generating the set of tactile vibration pulses, the set of audio pulses, or both, in accordance with a second respiration rate pattern that is based at least in part on the additional respiration rate data.

6. The method of claim 1, further comprising:
causing the graphical user interface of the user device to display a prompt for the respiration biofeedback session based at least in part on a calculated bedtime associated with the user, a circadian rhythm associated with the user, prior respiration biofeedback sessions associated with the user, or any combination thereof, wherein acquiring the physiological data during the respiration biofeedback session is based at least in part on the prompt.

7. The method of claim 6, further comprising:
receiving, via the user device, the wearable device, or both, a user input initiating the respiration biofeedback session, wherein the user input is received in response to the prompt, and wherein acquiring the physiological data, generating the set of tactile vibration pulses, the set of audio pulses, or both, is based at least in part on the user input.

8. The method of claim 7, further comprising:
causing the graphical user interface of the user device to display a message instructing the user to place their hand on the user device based at least in part on receiving the user input, wherein generating the set of tactile vibration pulses is based at least in part on displaying the message.

9. The method of claim 1, further comprising:
initiating the respiration biofeedback session based at least in part on additional physiological data acquired from the user, the additional physiological data comprising a heart rate, a respiration rate, one or more stress indicators, or any combination thereof, wherein acquiring the physiological data is based at least in part on initiating the respiration biofeedback session.

10. The method of claim 1, wherein the physiological data comprises temperature data, heart rate data, heart rate variability data, acceleration data, bioimpedance data, or any combination thereof, the method further comprising:
determining one or more parameters of the respiration rate pattern based at least in part on the temperature data, the heart rate data, the heart rate variability data, the acceleration data, bioimpedance data, or any combination thereof.

11. The method of claim 10, wherein the one or more parameters comprise a magnitude of the set of tactile vibration pulses, a volume of the set of audio pulses, a duration of each tactile vibration pulse of the set of tactile vibration pulses, a duration of each audio pulse of the set of audio pulses, a frequency of the set of tactile vibration pulses, a frequency of the set of audio pulses, or any combination thereof.

12. The method of claim 1, wherein the physiological data further comprises temperature data, and wherein the wearable device comprises a wearable ring device worn on a finger of a hand of the user, the method further comprising:
determining a temperature of the user device based at least in part on determining that the hand of the user is placed on the user device during the respiration biofeedback session; and
selectively modifying the temperature data associated with the user based at least in part on the temperature of the user device and based at least in part on determining that the hand of the user is placed on the user device during the respiration biofeedback session.

13. The method of claim 1, wherein the wearable device comprises a wearable ring device.

14. The method of claim 1, wherein the wearable device collects the physiological data from the user based on arterial blood flow.
